SEQUENCE LISTING (6 sequences)

SEQ ID NO: 1 - DNA, 22 bp, Artificial Sequence
  DNA 1--Thiolated ssDNA
ggaatacacgcgcgaaatcacg

SEQ ID NO: 2 - DNA, 22 bp, Artificial Sequence
  DNA 1--Unthiolated ssDNA
cgtgaattcgcgcgtgtattcc

SEQ ID NO: 3 - DNA, 27 bp, Artificial Sequence
  DNA 2--Thiolated ssDNA
aaaaaaatatataattaaaagttgaaa

SEQ ID NO: 4 - DNA, 27 bp, Artificial Sequence
  DNA 2--Unthiolated ssDNA
tttttttatatattaattttcaacttt

SEQ ID NO: 5 - DNA, 25 bp, Artificial Sequence
  DNA 3--Thiolated ssDNA
tggtggatggcgcagtcggcgacaa

SEQ ID NO: 6 - DNA, 12 bp, Artificial Sequence
  Dickerson′s dodecamer
cgcgaattcgcg

(19) United States
(12) Patent Application Publication
(10) Pub. No.: US 2020/0164072 A1
(43) Pub. Date: May 28, 2020

(54) NANOCOMPLEXES FOR REMOTELY-TRIGGERED GUEST MOLECULE RELEASE AND METHODS FOR FABRICATING THE SAME

(71) Applicant: William Marsh Rice University, Houston, TX (US)

(72) Inventors: Nancy J. Halas, Houston, TX (US); Oara Neumann, Houston, TX (US); Amanda M. Goodman, Houston, TX (US); Sandra W. Bishnoi, Houston, TX (US); Susan E. Clare, Houston, TX (US)

(73) Assignee: William Marsh Rice University, Houston, TX (US)

(21) Appl. No.: 16/595,060

(22) Filed: Oct. 7, 2019

Related U.S. Application Data
(60) Provisional application No. 62/742,122, filed on Oct. 5, 2018.

(51) Int. Cl. A61K 41/00; A61K 47/26; A61K 31/498; A61K 9/51; A61K 38/19; A61K 31/713; A61K 47/42; A61K 9/127; A61K 31/337; A61K 38/38

(52) U.S. Cl. CPC A61K 41/0042; A61K 47/26; A61K 31/498; A61K 9/5115; A61K 38/385; A61K 31/713; A61K 47/42; A61K 9/127; A61K 31/337; A61K 38/191

(57) ABSTRACT

A nanosample capable of near-infrared light-triggered release of therapeutic molecules. The nanosample includes a plurality of nanocomplexes. Each of the nanocomplexes includes a nanoshell; a host molecule linked to the nanoshell; and a guest molecule linked to the host molecule. The nanoshell includes a shell. The nanocomplex has a plasmon resonance wavelength. When irradiated with electromagnetic radiation of the plasmon resonance wavelength, plasmon resonance of the nanocomplex releases the guest molecule. The nanoshell may also include a core, where the shell surrounds the core. The nanoshell may be a nanomatryoshka. A link between the nanoshell and the host molecule may be a gold-thiol interaction. The shell may include at least one metal, such as gold or silver. The core may be a liposome and/or silica. The host molecule may be: synthetic polymers, biopolymers, polynucleotides, nucleic acids, polypeptides, polysaccharides, polyterpenes, lipids, aptamers, and/or proteins. The guest molecule may be: pharmaceutical molecules, biopharmaceutical molecules, oligonucleotides, nucleic acids, dye molecules, and/or imaging contrast agents. The host molecule may be: aptamer, single-stranded DNA, double-stranded DNA, and/or human serum albumin. The guest molecule may be: docetaxel, lapatinib, and/or tumor necrosis factor alpha. The plasmon resonance wavelength may be in a near-infrared (NIR) water window.

### CROSS-REFERENCE TO RELATED APPLICATIONS

This Application claims the benefit of U.S. Provisional Application 62/742,122 filed on Oct. 5, 2018.

### STATEMENT REGARDING FEDERALLY SPONSORED RESEARCH OR DEVELOPMENT

This invention was made with government support under Grant Number DGE-0940902 awarded by the National Science Foundation (NSF); Grant Number FA9550-15-1-0022 awarded by the Air Force Office of Scientific Research (USAF/AFOSR); and Grant Number W81XWH-13-1-0341, and Grant Number W81XWH-13-1-0342 awarded from the Army Medical Research and Material Command (ARMY/MRMC). The government has certain rights in the invention.

### SEQUENCE LISTING

The instant application contains a Sequence Listing entitled “US_16_595060 SequenceListing.txt” (1,519 bytes) and created on Jan. 13, 2020 which is submitted electronically via EFS-Web in ASCII format herewith and is hereby incorporated by reference in its entirety. The Sequence Listing, filed in accordance with 37 CFR 1.821(g), does not include new matter.

### FIELD OF THE DISCLOSURE

The present invention relates to bio-compatible nanostructures for remote release of a guest chemical and the methods for fabricating the same.

### BACKGROUND

Breast cancer is the leading cause of cancer deaths among women worldwide, accounting for 1.7 million new diagnoses in 2012. Current treatments for breast cancer include a combination of surgery, radiation, chemotherapy, molecularly targeted, and antihormonal therapeutics. One promising approach in cancer treatment is the use of gold-based nanostructures, whose strong optical absorption is due to their plasmon resonance, to provide safe and effective light-based therapeutics.

Plasmonic nanostructures are advantageous due to their unique optical properties, low toxicity, in vivo stability, and enhanced tumor uptake. However, existing approaches to treat cancer using plasmonic nanoparticles rely on photothermal heating of silica core-gold shell nanoshells (NS) to locally ablate tumors, causing nonspecific cell death.

Near-IR light has also been used to selectively release directly attached oligonucleotides and molecules from the plasmonic nanoparticle surface, which may occur at far lower average power. However, many existing U.S. Food and Drug Administration (FDA) approved drug molecules cannot be attached directly to the surface of plasmonic nanoparticles. Those that may be directly attached have a limited loading amount.

Finally, there are many FDA-approved drugs that are known to be highly effective but are currently underutilized because of low bioavailability and/or poor patient compliance due to adverse medication side effects. Additionally, when a drug is directly attached to a plasmonic nanoparticle surface, the drug directly interacts with the cells and, therefore, has the same low bioavailability and adverse side effects as the free drug.

The invention was also made with private support under Grant Number C-1220 awarded by the Robert A. Welch Foundation and Grant Number L-C-0004 awarded by the J. Evans Attwell-Welch Fellowship.

### SUMMARY

According to one or more embodiments, the flexible nanocomplex system and accompanying fabrication method described herein allows for the indirect attachment of a large number of existing therapeutic molecules to a plasmonic nanoshell whereby the therapeutic molecules may be released via irradiation with electromagnetic radiation. Furthermore, such a system will allow for the remotely-triggered release of a high local drug concentration within a spatially localized region (e.g., the site of a tumor or metastatic disease), while providing a relatively low systemic burden for a patient. Thus, this approach unleashes the potential of known, highly-effective drugs that would otherwise be toxic at high systemic doses. Finally, these nanocomplexes can provide efficient drug delivery into cells, thereby provoking less of an immune response compared with free drug molecules. When used for drug delivery, the nanocomplex may result in treatments with less toxicity, more compliance, and greater disease control.

In one or more embodiments, the flexible fabrication and release strategies shown here will allow release to be more easily controlled and tailored for various chemotherapeutic drugs and cancer types. This in turn has the potential for increasing therapeutic efficacy by delivering high local drug concentrations while keeping systemic drug concentrations low, which would reduce the well-known deleterious side effects of conventional chemotherapeutic drug delivery methods.

While both the extracellular and intercellular spaces of the body contain water, many existing FDA-approved drugs are hydrophobic. This hydrophobicity often leads to poor adsorption, low bioavailability, short blood circulation time, low cellular uptake, and increased immune response. Each of these leads to poor patient compliance for drugs that would otherwise be effective. One or more embodiments of the present disclosure can be used to deliver therapeutic molecules controllably and minimize the systemic toxicity induced by the administration of hydrophobic drugs by conventional means. Embodiments herein may advantageously use proteins or DNA as a scaffold (i.e., a host molecule) to deliver guest molecules, such as chemotherapy drug molecules. The advantage is that proteins and DNA are biologically compatible scaffolds that can deliver a wide range of hydrophobic or hydrophilic drug molecules. Host-conjugated nanoparticles according to one or more embodiments may allow the drug molecule to be surrounded by the host molecule until its controlled release, which may provide efficient internalization into cells, increased cellular uptake, improved blood circulation time, and provoke less of an immune response than free drug molecules. For example, by creating a host-conjugated nanoparticle, a hydrophobic drug may be carried by a hydrophilic host until release according to one or more embodiments of the disclosure.

Furthermore, depending upon the drug molecule, the type of host molecule, and the laser illumination method (continuous wave or pulsed laser), in vitro light-triggered release can be achieved, according to one or more embodiments. This nanohost delivery vector may maximize the drug loading, preserve the guest molecule by minimizing non-desired interactions with other molecules, and provide light-triggered release with controllable delivery with increased drug loading.

The biological half-life of a florescent dye is typically on the order of 5 hours. However, when incorporated into a nanomatryoshka (NM), the half-life may be 24 hours or longer. Furthermore, employing a NM at the center of a nanocomplex with such an added functionality may not affect the drug release capabilities of the nanocomplex, and while adding additional functionality, such as optical tracking. According to one or more embodiments, encapsulation of dye molecules and metal ions simultaneously between an inner core and an outer shell in a multilayered geometry of a NM will give us the ability to study the quantitative tracking of therapeutic nanoparticles in vivo, investigate nanoparticle biodistribution, and cellular processes via magnetic resonance imaging (MRI) and fluorescence microscopy. This evaluation will determine how much drug molecules will be necessary for an effective treatment and correlate the amount of drug molecules with the tumor size.

The flexible loading and release strategies, according to one or more embodiments, will allow release to be more easily controlled and tailored for various chemotherapeutic drugs and cancer types. Several intercalated molecules released from NM or NS nanoparticle delivery vectors are quantifiable and it will be possible to use the delivered molecules to study cellular processes which are either independent or dependent on concentration. Diffusion of released molecules inside cells can also be studied. Immediately after the controlled release mechanism is applied, the released molecule (or molecules) can be followed as it diffuses through the cell.

Embodiments herein may provide for controlled release. The controlled release of a molecule within a living cell has broad applications for when it is necessary to control the time when the host intercalating molecule is released and control the location in which the host intercalating molecule is released.

Embodiments herein may also provide for monitored cellular cytotoxicity resulting from light-triggered release of highly toxic hydrophobic chemotherapeutic drugs using MRI, Fluorescence, or Bright field measurements.

The linking of lapatinib (LAP) to human serum albumin (HAS) deserves specific comment as an illustrative application. Tyrosine kinase inhibitors, such as LAP, are notoriously insoluble in water, which is one of the reasons why this class of drug has poor oral bioavailability. Additionally, although clinical trials have demonstrated that the combination of LAP with other human epidermal growth factor receptor 2 (HER2)-directed therapies for dual blockade may benefit certain subgroups of patients, severe toxicities, diarrhea in particular, lead to poor compliance and treatment discontinuation. However, HSA is the most abundant protein in blood plasma and can bind highly insoluble drug molecules in its hydrophobic pockets. Therefore, the NS @HSA@LAP formulation according to embodiments herein delivers an efficacious dose of a poorly soluble drug directly to the tumor where it can be released. As it will be released only at the tumor, the systemic dose will be less than with current oral formulations resulting in less toxicity, more compliance, and greater disease control. Finally, due to its large size and multiple binding regions, a HAS-containing nanocomplex may have a significant guest molecule capacity.

Finally, the same flexible loading and release strategies may be used to transport drugs for release within a localized region to treat conditions other than cancer, according to the one or more embodiments described herein. Additionally, these strategies may be used to transport and release molecules that are not traditional, small molecule drugs, such as imaging contrast agents, dye molecules, gene therapy agents, biologic agents, etc.

One or more embodiments may describe a nanosample that includes a plurality of nanocomplexes. Each nanocomplex may include a nanoshell; a host molecule linked to the nanoshell; and a guest molecule linked to the host molecule. One or more embodiments of the nanoshell may include a shell. According to one or more embodiments, the nanocomplex may have a plasmon resonance wavelength.

One or more embodiments may describe a nanosample capable of near-infrared light-triggered release of therapeutic molecules. The nanosample, according to one or more embodiments, may include a plurality of nanocomplexes. Each of the nanocomplexes may include a nanoshell; a host molecule linked to the nanoshell; and a guest molecule linked to the host molecule. One or more embodiments of the nanoshell may include a shell. According to one or more embodiments, the nanocomplex may have a plasmon resonance wavelength. In one or more embodiments, when irradiated with electromagnetic radiation of the plasmon resonance wavelength, plasmon resonance of the nanocomplex may release the guest molecule.

In one or more embodiments, the nanoshell may also include a core, where the shell surrounds the core.

In one or more embodiments, the nanoshell may be a nanomatryoshka.

In one or more embodiments, a link between the nanoshell and the host molecule may be a gold-thiol interaction.

In one or more embodiments, the shell may comprise at least one metal.

In one or more embodiments, the core may comprise liposome and/or silica; and the shell may be gold and/or silver.

In one or more embodiments, the host molecule may include least one of synthetic polymers, biopolymers, polynucleotides, nucleic acids, polypeptides, polysaccharides, polyterpenes, lipids, aptamers, and/or proteins, or combinations thereof.

In one or more embodiments, the guest molecule may include at least one of pharmaceutical molecules, biopharmaceutical molecules, oligonucleotides, nucleic acids, dye molecules, imaging contrast agents, or combinations thereof.

In one or more embodiments, the host molecule may be selected from tumor necrosis factor alpha (TNF-α), single-stranded DNA (ssDNA), double-stranded DNA (dsDNA) or human serum albumin (HAS).

In one or more embodiments, the guest molecule may be selected from docetaxel (DTX), lapatinib (LAP), and TNF-α.

In one or more embodiments, the host molecule may be dsDNA and the guest molecule may be DTX.

In one or more embodiments, the host molecule may be HAS and the guest molecule may be LAP.

In one or more embodiments, the host molecule may be TNF-α aptamer and the guest molecule may be TNF-α.

According to one or more embodiments, a method for fabricating a nanosample capable of near-infrared light-triggered release of therapeutic molecules may include: synthesizing a nanoshell; functionalizing a surface of the nanoshell with a host molecule to form a nanohost; incubating the nanohost with a guest molecule to form a nanocomplex; and sterilizing the nanocomplex and forming the nanosample.

According to one or more embodiments, a method for fabricating a nanosample capable of near-infrared light-triggered release of therapeutic molecules may further include: preparing the host molecule for functionalization of the nanoshell. Preparing the host molecule for functionalization may include: reducing the disulfide bonds of two ssDNA samples, and mixing the two ssDNA samples to form dsDNA. According to one or more embodiments, the host molecule may be the dsDNA, and the two ssDNA samples may have complementary sequences.

One or more embodiments may disclose a nanosample capable of near-infrared light-triggered release of therapeutic molecules for treating cancer. According to one or more embodiments, the nanosample may include a plurality of nanocomplexes; a host molecule linked to the nanoshell; and a guest molecule linked to the host molecule. Some embodiments of the nanoshell may include a shell that may include a metal; and a core that may include a non-metal. In some embodiments, the host molecule may be selected from aptamer, ssDNA, dsDNA, or HAS. In some embodiments, the guest molecule may be a pharmaceutical molecule or a biopharmaceutical molecule. In some embodiments, the nanocomplex may have a plasmon resonance wavelength in a near-infrared (NIR) water window. According to some embodiments, the nanocomplex may release the guest molecule when irradiated with electromagnetic radiation of the plasmon resonance wavelength.

### BRIEF DESCRIPTION OF DRAWINGS

FIG. 1 is a schematic of a nanocomplex, according to one of more embodiments.

FIG. 2A-2D are schematic representations, according to one or more embodiments, of intermediate compounds generated during formation of a nanosample.

FIGS. 3A and 3B are schematic representation of a nanosample during and after exposure to electromagnetic radiation, according to one or more embodiments.

FIG. 4A-4C are transmission electron microscopy (TEM) images of an NS @DNA2@LAP nanocomplex and its intermediate compositions, according to one or more embodiments.

FIG. 5A-5C are TEM images of an NS @HS @LAP nanocomplex and its intermediate compositions, according to one or more embodiments.

FIG. 6A-6C depict surface-enhanced Raman spectroscopy (SERS) spectra of the nanocomplexes NS @DNA1@DTX, NS @DNA2@LAP, and NS @HS @LAP, compared with their intermediate compositions, according to one or more embodiments.

FIGS. 7A and 7B depict the cytotoxicity of NS @DNA1@DTX and NS @HSA@LAP in breast cancer cells with and without laser irradiation, according to one or more embodiments.

### DETAILED DESCRIPTION

Specific embodiments will now be described in detail with reference to the accompanying figures. In the following description, numerous details are set forth to provide an understanding of the present disclosure. However, it will be understood by those skilled in the art that the embodiments of the present disclosure may be practiced without these details and that numerous variations or modifications from the described embodiments may be possible.

Used herein, an “@” may be used to indicate linked components of the form: Nanoshell@HostMolecule@GuestMolecule. Hence, NS @dsDNA@DTX may describe a nanocomplex comprised of a NS, having double stranded DNA (dsDNA) linked to the NS, and having docetaxel (DTX) linked to the dsDNA.

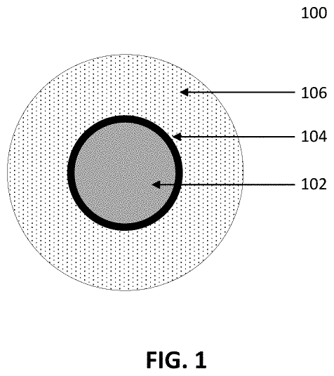

FIG. 1 depicts a single nanocomplex 100. Each nanocomplex is a multi-component nanostructure that may include a nanoshell; a host molecule that is linked to the nanoshell forming a nanohost; and a guest molecule that is linked to the host molecule forming the nanocomplex. Thus, in some embodiments, the host molecule may serve as a bridge or connection between the nanoshell and the guest molecule. Finally, a nanosample is a plurality of nanocomplexes.

At the center of each nanocomplex may be the nanoshell. The nanoshell may be composed of two components: a metallic shell 104 and a non-metallic core 102. In some embodiments, the non-metallic core 102 may be removed before any further steps, leaving only an empty metallic shell 104. In some embodiments, the nanoshell may be a nanomatryoshka with a metallic shell and more than one inner layer. Linked to a surface of the nanoshell is the nanohost that may also be linked to the guest molecule 106.

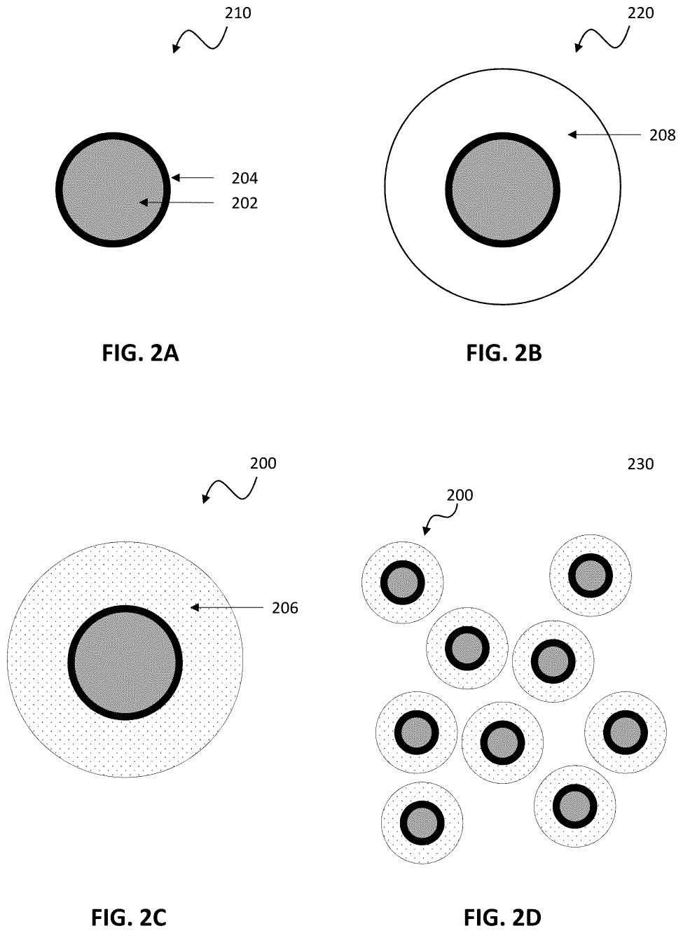

Broadly, according to one of more embodiments, a nanosample may be produced using a four-step process, including: synthesizing a nanoshell; functionalization of a surface of the nanoshell with a host molecule to form a nanohost; incubating the nanohost with the guest molecule to form a nanocomplex; and sterilizing and concentrating the nanocomplex to form a nanosample. Accordingly, this method of forming a nanosample and each of these constituent components is depicted in FIGS. 2A-2D and described in detail below.

At the center of each of the nanocomplexes is a nanoshell. FIG. 2A depicts a nanoshell 210, according to one or more embodiments. Nanoshell 210 has a metallic shell 204 and a non-metallic core 202. Thus, according to one or more embodiments, FIG. 2A depicts the nanoshell after completion of the first step of the above method (i.e., “synthesizing a nano shell).

In one or more embodiments, the metallic shell 204 may be formed from any metal or combination of metals (e.g., alloys), including gold, silver, platinum, nickel, palladium, ruthenium, rhodium, iridium, osmium, tantalum, copper, aluminum, titanium, vanadium, iron, cobalt, chromium, europium, erbium, niobium, nitinol, and stainless steel. For some applications, for example biological applications, the materials selection for the metallic shell may be informed by features such as its environmental compatibility or biocompatibility.

In one or more embodiments, the non-metallic core 202 may be a biomolecule (e.g., liposome, cellulose, lipid, polylysine, etc.), a dielectric material (e.g., SiO₂, TiO₂, Al₂O₃, ZrO₂, FePO₄, glass, ceramic, etc.), or a polymer (e.g., polyethylene, silicone, polyester, poly(methyl methacrylate), polyanhydride, polydimethylsiloxane, polydioxanone, polyethylene glycol, polyethylene terephthalate, polyglycolide, polyhydroxyalkanoates, polyimide, polytetrafluoroethylene, polyvinylidene fluoride, etc.). For some applications, for example biological applications, the material selection for the non-metal core may be informed by features such as its environmental compatibility or biocompatibility. Some embodiments of the nanoshell may lack a present core because it has been removed. Therefore, the metallic shell used to create the nanocomplex may surround empty space instead of a non-metallic core in one or more embodiments. Furthermore, according to some embodiments, the non-metal core may be hollow. In one or more embodiments having a hollow non-metal core, the non-metal core may further be filled with therapeutic agents, dye molecules, imaging contrast agents, etc. to add additional functionality.

In one or more embodiments, the nanoshell may be spherical, rod shaped, star shaped, or a cubical, or any other shape that has a suitable plasmon resonance.

A plasmon resonance is a collective oscillation of the conduction band electrons within a metal surface upon excitation with an external electromagnetic field. In one or more embodiments, the nanoshell may support plasmon resonance, which may be used to remotely release guest molecules from the nanocomplexes.

Many factors may impact the plasmon resonance of a nanocomplex, including the geometry of and the material of each constituent component. The largest factor may be the nanoshell plasmon resonance, which may be largely dictated by the nanoshell geometry, such as the core-shell dimensions. The geometries and materials of the host molecule and the guest molecule also influence the plasmon renounce, although their impact may be smaller than the influence of the nanoshell. When nanoshells are aggregated or clumped, the plasmon resonance may change and/or be dampened. However, the addition of the host and guest molecules around the nanoshells in the nanocomplex serves to separate the nanoshells and provide chemical stability in time. Furthermore, when the nanosample is monodispersed like when it is diluted within the body the plasmon resonance of the nanosample is equivalent to the plasmon resonance of each nanocomplex. Accordingly, in one or more embodiments, the plasmon resonance of the nanosample may be equal to the plasmon resonance of one nanocomplex.

Furthermore, said features of the nanocomplex may be controlled so this plasmon resonance occurs at a desirable wavelength for a particular application. To produce a plasmon resonance at a particular wavelength, the geometry of the nanoshell may vary depending upon the materials selected for the metallic shell and the non-metallic core.

In some embodiments, the plasmon resonance of the nanocomplex may be in the infrared (IR) regime. The IR regime may be defined as a wavelength between 650 nm and 1 mm.

In some embodiments, the plasmon resonance of the nanocomplex may be in the near-infrared (NIR) regime. The NIR regime may be defined as a wavelength between 650 nm and 2,500 nm.

The NIR spectral water window is the range of wavelengths where light has a maximum penetration depth in biological tissue. Thus, in some embodiments, the nanoshell plasmon resonance may be tuned to be within a NIR spectral water window where the biological matter has the highest transparency. Three NIR spectral water windows exist: NIR-I (700-950 nm), NIR-II (1000-1350 nm), and NIR-III (1550-1870 nm).

In one or more embodiments, the nanoshell may be a nanomatryoshka (NM). A nanomatryoshka is a nanoparticle with multiple layers. For example, the nanomatryoshka may comprise a metallic core (e.g., Au), an interstitial nanoscale non-metallic layer (e.g., SiO₂), and a metallic shell (e.g., Au), although alternative nanomatryoshkas may have a larger number of layers. In one or more embodiments, the nanomatryoshka within the nanocomplex may have any number of layers if a nanohost is able to be attached. Thus, according to some embodiments, because a nanomatryoshka may have both plasmon resonance and an outermost shell that is metallic, it may be used as the nanoshell from which a nanocomplex may be developed. Furthermore, the diameter of a nanomatryoshka may be smaller than the diameter of a nanoshell made of the same materials with the same plasmon resonance wavelength. Since the cellular uptake of nanoparticles may increase as the nanoparticle diameter decreases, one may employ a nanomatryoshka as the nanoshell to produce a nanocomplex with superior cellular uptake. Furthermore, in some embodiments, a resonant nanoshell with a smaller diameter, such as a NM, may have a smaller cross section and thus may have a larger, local increase in temperature than a nanoshell with a larger diameter.

Many NMs have been developed that incorporate additional materials within, for example, the dielectric to perform some function in addition to phonon resonance. For example, NMs may incorporate a Ti magnetic resonance imaging (MRI) and/or fluorescent contrast agents such as chelated metal ions (e.g., Fe³⁺, Mn²⁺, Gd³⁺, other rare earth metal ions, etc.) and/or fluorescent dyes (e.g., IRDYE 800, CY5, CY7, etc.). In some embodiments, these additional materials may be incorporated into the dielectric layer. For these NMs, this additional functionality does not impact the external metallic shell. Accordingly, in embodiments herein, the NM with additional functionality may be employed as the nanoshell at the center of the nanocomplexes.

Nanocomplexes formed around a spherical nanoshell with a 120 nm diameter SiO₂ core surrounded by a 10-15 nm Au shell having a plasmon resonance within the NIR water window of around 808 nm are described herein as a non-limiting example, according to one or more embodiments.

A nanocomplex further includes a host molecule that is linked to a nanoshell, such as linked to a surface of the nanoshell, to form a nanohost. FIG. 2B depicts the nanohost 220 after the host molecule 208 has been linked to the nanoshell, according to one or more embodiments. Thus, according to one or more embodiments, FIG. 2B depicts the nanohost 220 after completion of the second step above (i.e., “functionalization of a surface of the nanoshell with a host molecule to form a nanohost”).

In some embodiments, the host molecule may be readily conjugated for attachment to the surface of the nanoshell. Additionally, in some embodiments, the structures of the host molecule may also be tailorable for uptake of guest molecules in a host-guest manner. Thus, in view of the final nanocomplex, the host molecule may serve as a bridge between the nanoshell and the guest molecule. Additionally, the host molecule thus may be able to form a link with both the nanoshell and a guest molecule.

In some embodiments, the link between the nanoshell and the host molecule may be a chemical bond, of any type. In some embodiments, this link is a bond which may be covalent, ionic, hydrogen, electrostatic, π-π interaction, or other. In some embodiments, this link is due to a chemical bond formed via a gold-thiol interaction. The gold-thiol interaction is a very strong bond. Additional bonding groups may be used, such as amine, amide, acid, carboxyl. peptidic groups, etc.

In some embodiments, the host molecule may be a polymer. In some embodiments, said polymer may be a biopolymer or a synthetic polymer. According to some embodiments, the natural or synthetic polymer host molecule may be linear, dendritic, networked, branched, and/or crosslinked. In some embodiments, the host molecule may include two or more host molecules that may be miscible, immiscible, and/or compatible co-polymers.

In some embodiments, that biopolymer may be a polynucleotide, nucleic acid, polypeptide, polysaccharide, polyterpene, lipid, and/or protein, and combinations thereof.

Here, polynucleotide, polypeptide, polysaccharide, and polyterpene are used to describe a biopolymer regardless of the number of nucleotides, amino acids, monosaccharides, and isoprenes, respectively, in the final molecule. Put another way, the group of polypeptides also includes oligopeptides formed of any number of amino acids in the chain and any other biopolymer formed of amino acids. The terms polynucleotide, polysaccharide, and polyterpene are similarly broad.

In some embodiments, the host molecule may be a polynucleotide. According to one or more embodiments, the host molecule may be a polynucleotide in the form of a nucleic acid aptamer, such as a DNA aptamer, an RNA aptamer, and/or a nucleic acid analogue (XNA) aptamer.

In some embodiments, the host molecule may be a nucleic acid. A few examples of nucleic acid host molecules, according to one or more embodiments, may be DNA, RNA, XNA, or an artificial nucleic acid. A person having skill in the art will appreciate that the exact sequence of the nucleotides within the nucleic acid and any additional functionalization groups added to the nucleic acid may be adapted for the particular nanoshell and guest molecule used, as well as the intended application.

In some embodiments, the host molecule may be a polypeptide. An example of a polypeptide host molecule, according to one or more embodiments, may be a polypeptide such as a peptide aptamer.

In some embodiments, the host molecule may be a polysaccharide. A few examples of polysaccharide host molecules, according to one or more embodiments, may be glucose, fructose, cellulose, sucrose, maltose, lactose, and glycogen.

In some embodiments, the host molecule may be a polyterpene. An example of a polyterpene host molecule, according to one or more embodiments, may be natural rubber.

In some embodiments, the host molecule may be a lipid. A few examples of lipid host molecules, according to one or more embodiments, may be fatty acids, antibodies, glycerolipids, phospholipids, sphingomyelins, sterols, prenols, saccarolipids, and polyketides.

In some embodiments, the host molecule may be a protein. A few examples of protein host molecules, according to one or more embodiments, may be either natural-body proteins or artificial proteins. Specifically, protein host molecules may include artificial poly(A)-binding protein, human serum albumin (HAS), ovalbumin, and bovine serum albumin.

In some embodiments, the host molecule may be a synthetic polymer having any number of monomers. A few examples of synthetic polymer host molecules, according to one or more embodiments, may be polyethylene, silicone, polyester, poly(methyl methacrylate), polyanhydride, polydimethylsiloxane, polydioxanone, polyethylene glycol, polyethylene terephthalate, polyglycolide, polyhydroxyalkanoate, polyimide, polytetrafluoroethylene, and polyvinylidene fluoride.

The final component of each of the plurality of nanocomplexes is a guest molecule. The guest molecules are the molecules that may be released from the nanocomplex when the nanocomplex undergoes plasmon resonance as a result of irradiation with electromagnetic radiation. The guest molecule may be linked to the nanoshell indirectly through a link with the host molecule.

FIG. 2C depicts one or more embodiments of the nanocomplex 200 where the host molecule has been modified by linkage to the guest molecule 206. Thus, according to one or more embodiments, FIG. 2C depicts the nanocomplex after completion of the third step above (i.e., “incubating the nanohost with the guest molecule to form a nanocomplex”). Accordingly, the nanohost linked to the guest molecule 206 is depicted differently in FIG. 2C than the nanohost before it is linked to the guest molecule 208 in FIG. 2B.

In some embodiments, the link between the nanohost and the guest molecule may be a chemical bond, of any type. In some embodiments, this link is a bond which may be covalent, ionic, hydrogen, or other. The guest molecule may, in some embodiments, be bonded to the host molecule by hydrogen bonding electrostatic, π-π interaction, or van der Waals forces. In some embodiments, the bond between the host molecule and the guest molecule may be weaker than the bond between the host molecule and the nanoshell. In some embodiments, the guest molecule may be intercalated into the host molecule, such as within the strands of a dsDNA host molecule or within the folds of a protein host molecule.

In some embodiments, a ratio between guest molecules and host molecule may be greater than 0.25 guest molecules per host molecule (i.e., greater than 0.5 guest molecules per host molecule, greater than 0.75 guest molecules per host molecule, greater than 1 guest molecule per host molecule, greater than 2 guest molecules per host molecule, greater than 3 guest molecules per host molecule, greater than 5 guest molecules per host molecule, greater than 10 guest molecules per host molecule, etc.). In some embodiments, the ratio between guest molecules and host molecule may be between 0.25 and 20 guest molecules per host molecule.

In some embodiments, the guest molecule is linked to the host molecule by incubation of the host molecule with the guest molecule. In some embodiments, the guest molecule and the host molecule may be combined in a solution and allowed to incubate for an incubation period. This incubation period may be greater than 0.25 h (e.g., greater than 0.5 h, greater than 0.75 h, greater than 1 h, greater than 2 h, greater than 3 h, greater than 4 h, greater than 8 h, greater than 12 h, greater than 18 h, greater than 24 h, etc.). In some embodiments, the incubation period may be between 0.25 h and 48 h.

During incubation, the solution may be agitated, stirred, shaken, etc. so the constituents are well mixed.

Various types of guest molecules may be incorporated into the nanocomplex for a wide variety of purposes. In one or more embodiments, the nanocomplex may include one or more types of guest molecules, such as a combination of 2, 3, 4, or more types of guest molecules. The guest molecule may be small molecule drugs (i.e., have a molecular weight less than 900 Da) and/or biologic medical products (e.g., having a molecular weight greater than 900 Da). The guest molecule may be derived from chemical synthesis and/or may be a biologic medical product.

The guest molecule may be a biomolecule such as a pharmaceutical molecule, a biopharmaceutical molecule, an oligonucleotide, a nucleic acid, an imaging contrast agent, a dye molecule, etc.

In some embodiments, the guest molecule may be a pharmaceutical molecule. A pharmaceutical guest molecule, according to one or more embodiments, may be any molecule within any of the classes of the Anatomical Therapeutic Chemical (ATC) Classification System, or a new molecule which is not yet classified. Therefore, for example, the pharmaceutical guest molecule may be within any of the first level groups of the ATC classification system: alimentary tract and metabolism, blood and blood forming organs; cardiovascular system; dermatologicals; genito-urinary system and sex hormones; systemic hormonal preparations, excluding sex hormones and insulins; antiinfectives for systemic use; antineoplastic and immunomodulating agents; musculo-skeletal system; nervous system; antiparasitic products, insecticides and repellents; respiratory system; sensory organs; and various, or a combination thereof.

In some embodiments, the pharmaceutical guest molecule may be within the antineoplastic and immunomodulating agents group, which are commonly referred to as chemotherapy agents or chemotherapy drugs.

In some embodiments, the guest molecule may be a biopharmaceutical molecule. A few examples of classes of biopharmaceutical guest molecules, according to one or more embodiments, may be a biologic agent, a gene therapy, a vaccine, a recombinant DNA, insulin, a cell therapy agent, a hormone, an antibody, a blood factor, a thrombolytic agent, a hematopoietic growth factor, an interferon, an interleukin-based product, a therapeutic enzyme, a tumor necrosis factor, or a combination thereof.

In some embodiments, an antibody guest molecule may aide in targeting the correct cell type by increasing the accumulation at a target site. An aptamer, which may function as an artificial antibody, may behave as a host molecule in a similar fashion.

Finally, in some embodiments, an additional guest molecule may be attached to the NS along with a small molecule drug and/or biologic medical product. This additional guest molecule may be used for modifying pharmacogenetic parameters (e.g., surfactants), enhancing the nanocomplex stability (e.g., poly-L-lysine), prolonging the circulation half-life (e.g., PEG), slowing the drug release (e.g., cationic polymers), or as a targeting agent (e.g., folate, thermosensitive polymers, transferrin, apolipoproteins, and monoclonal antibodies).

Finally, FIG. 2D depicts a nanosample 230, which comprises a plurality of nanocomplexes 200. A nanosample, containing a plurality of nanocomplexes, may have increased therapeutic utility compared with a single nanocomplex. Thus, according to one or more embodiments, FIG. 2D depicts the nanosample after completion of the fourth step above (i.e., “sterilizing the nanocomplex and forming the nanosample”).

The nanosample may include a plurality of nanocomplexes. The nanosample may be in a liquid form, in a solid form (e.g., a powder), may be arrayed on a substrate (e.g., a Langmuir-Blodgett film), or may be incorporated into a composite (e.g., dispersed in a polymer).

In the liquid form, the nanosample may be diluted in a solvent such that the nanocomplexes and the solvent form a solution, a colloid, a colloidal suspension, or a suspension. The composition of the solvent and the final concentration of the nanosample depend on the intended application. The solvent may be sterile.

In some embodiments, the nanocomplex solution may be sterilized by being passed through a biological cellular filter. The nanocomplex solution may be diluted before being passed through the filter.

In some embodiments, the nanocomplex solution may be centrifuged to concentrate the nanocomplex solution and/or to remove the nanocomplex solvent.

According to one or more embodiments, a nanosample may be a solution of a plurality of nanocomplexes in a solvent, forming a colloidal suspension. In some embodiments, the concentration of the nanosample may be high. In some embodiments, a concentration of the nanosample may be less than 10 mg of metal/mL (e.g., less than 5 mg of metal/mL, less than 3 mg of metal/mL, less than 2 mg of metal/mL, less than 1.5 mg of metal/mL; less than 1 mg of metal/mL, etc.). In some embodiments, the concentration of the nanosample may be between 0.01 mg of metal/mL and 20 mg of metal/mL. In some embodiments, the concentration of the nano sample may be between 0.1 mg of Au/mL and 20 mg of Au/mL.

The nanosample may be a colloidal suspension of the plurality of nanocomplexes in a solvent, according to one or more embodiments. The solvent, in some embodiments, may be a sterile buffer solution or cellular media. In some embodiments, the solvent may be 1 mM phosphate buffer, pH 7.3.

Release of Guest Molecule Using Electromagnetic Irradiation

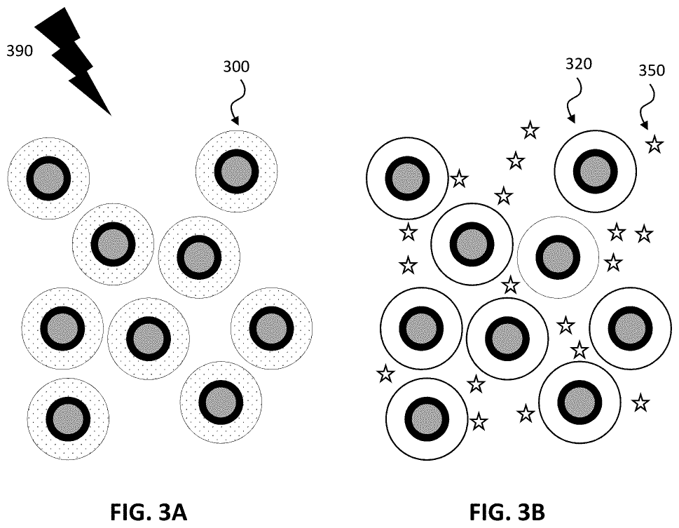

FIGS. 3A and 3B are schematic representations of a nanosample before and after irradiation with electromagnetic radiation, respectively. FIG. 3A depicts the nanosample, comprised of a plurality of nanocomplexes 300 which have linked guest molecules, as it is irradiated with electromagnetic radiation 390. FIG. 3B depicts the nanosample, after irradiation, which is comprised of the plurality of nanocomplexes 320 which have released the guest molecules 350. For remotely triggered guest molecule delivery, the nanocomplexes drug release mechanism via plasmon resonance of the nanocomplexes may be important.

Release of the guest molecule may not release all the guest molecules that were incorporated into the nanocomplexes via the host molecule. In some embodiments, only a fraction of the guest molecules originally incorporated into the nanosample may be released upon irradiation such that they can interact. Upon irradiation with electromagnetic radiation, release of a fraction of the guest molecule from the nanocomplex may still be considered release. A remaining fraction of the guest molecules may continue to be linked to the host molecule and/or nanocomplex, unable to interact as intended. Put another way, release may be considered successful even if, after release, some fraction of the guest molecules remain unable to interact with their environment as intended.

As discussed above, nanocomplexes according to embodiments of this disclosure hay have a characteristic plasmon resonance wavelength. When excited by sufficiently energetic electromagnetic radiation of this wavelength, the nanocomplex may release a guest molecule via one or more mechanisms discussed below. To maximize drug-release efficiency of one or more embodiments, the exciting electromagnetic radiation may be either supplied by a continuous-wave (CW) laser or by a pulsed laser.

A CW laser is one whose output power is constant over time. For NS @dsDNA, CW-induced, light-triggered release requires the bulk temperature of a nanohost to rise above a thiolated dsDNA dehybridization temperature. Once the temperature has risen above the thiolated dsDNA dehybridization temperature, CW irradiation may cause non-thiolated single stranded DNA (ssDNA) to uncoil from the thiolated ssDNA that may still be attached to the NS via the Au—S bond. Therefore, dehybridization of the dsDNA may cause the release of the non-thiolated ssDNA host molecules along with the guest molecules. In some embodiments, the released ssDNA host molecules may serve as gene therapy agents or have other therapeutic purposes.

Thus, according to one or more embodiments, remotely triggering guest molecule release using a CW laser irradiation may involve irradiating a nanosample with a CW laser having a CW laser wavelength within one of the NIR water windows (i.e., NIR-I (700-950 nm), NIR-II (1000-1350 nm), and NIR-III (1550-1870 nm). In some embodiments, the CW laser wavelength may be 808 nm.

In addition to having a wavelength that matches the plasmon resonance of the nanocomplex, the CW laser must also impart sufficient power to release the guest molecules. According to one or more embodiments, remotely triggering guest molecule release using a CW laser irradiation may involve irradiating a nanosample with a CW laser imparting a CW power of at least 500 mW (e.g., at least 1.0 W, at least 1.5 W, at least 2.0 W, at least 2.5 W, at least 3.0 W, at least 5.0 W, at least 10 W, at least 20 W, etc.). In some embodiments, the CW power may be between 500 mW and 40 W. In some embodiments, optimizing the CW laser power may also include lowering the CW power to a power above a CW laser threshold power that is still effective at inducing plasmon resonance of the nanocomplex, in order to minimize the photothermal heating that may cause non-specific cell death or protein/enzyme coagulation/aggregation.

According to one or more embodiments, remotely triggering guest molecule release using CW irradiation may involve irradiating a nanosample with a CW laser for a CW irradiation duration greater than 30 seconds (e.g., greater than 1 min., greater than 1.5 min., greater than 2 min., greater than 2.5 min., greater than 5 min., greater than 10 minutes, greater than 20 minutes, etc.). In some embodiments, the CW laser may have a CW irradiation duration of between 30 seconds and 30 minutes.

According to one or more embodiments, the CW power and the CW irradiation duration may be inversely correlated. One of ordinary skill in the art may optimize the CW power and CW irradiation duration so as to properly trigger guest molecule release while minimizing the photothermal heating that may cause non-specific cell death. Furthermore, the CW laser power and the CW laser irritation duration may vary during imaging vs. during guest molecule release.

A pulsed laser is a laser where that the output power appears in pulses of some duration at some repetition rate. A pulsed laser scans through a large number of wavelengths, so may induce plasmon resonance in a nanocomplex by repeatedly imparting electromagnetic radiation that matches the resonance wavelength. For NS @dsDNA, pulsed laser-induced, light-triggered release using femtosecond near-IR laser pulses breaks the Au—S bond that binds a host molecule to the nanoparticle with no measurable bulk temperature increase. Since the temperature has not risen above the thiolated dsDNA dehybridization temperature, pulsed laser irradiation may cause the release of the dsDNA host molecules from the NS, and the dsDNA may still be attached to the guest molecules. For NS @HSA (and for other host molecules that are proteins or other biomolecules), the plasmon resonance of the NS may cause the Au—S bond to break, releasing the guest molecule from the NS. Once released, the guest molecule may change confirmation and unfold, which may release the guest molecule.

According to one or more embodiments, remotely triggering guest molecule release using pulsed laser irradiation may involve irradiating a nanosample with a pulsed laser having a pulse duration less than 1 nanosecond (e.g., less than 750 picoseconds, less than 500 picoseconds, less than 250 picoseconds, less than 1 picoseconds, less than 750 femtoseconds, less than 500 femtoseconds, less than 250 femtoseconds, less than 100 femtosecond, less than 50 femtosecond, less than 25 femtosecond, less than 10 femtosecond, etc.). In some embodiments, the pulse duration may be between 5 femtoseconds and 1 nanosecond.

According to one or more embodiments, remotely triggering guest molecule release using pulsed laser irradiation may involve irradiating a nanosample with a pulsed laser having an operating frequency of greater than 50 kHz, (e.g., greater than 100 kHz, greater than 250 kHz, greater than 500 kHz, greater than 750 kHz, greater than 1000 kHz, etc.). In some embodiments, the operating frequency may be between 50 kHz and 2000 kHz.

According to one or more embodiments, remotely triggering guest molecule release using pulsed laser irradiation may involve irradiating a nanosample with a pulsed laser having a pulsed laser output power of greater than 10 mW (e.g., greater than 25 mW, greater than 50 mW, greater than 75 mW, greater than 100 mW, greater than 200 mW, etc.). In some embodiments, the pulsed laser output power may be between 10 mW and 500 mW.

According to one or more embodiments, remotely triggering guest molecule release using pulsed laser irradiation may involve irradiating a nanosample with a pulsed laser for a pulsed laser irradiation duration greater than 5 seconds (e.g., greater than 10 seconds, greater than 30 seconds, greater than 1 min., greater than 1.5 min., greater than 2 min., greater than 2.5 min., greater than 5 min., etc.). In some embodiments, the pulsed laser irradiation duration may be between 1 second and 30 minutes.

Pulse-laser-induced guest molecule release may require lower average power than continuous-wave-induced guest molecule release. Higher average power may cause more photothermal heating, which may cause unspecific cell death. Therefore, in one or more embodiments, pulsed-laser-induced guest molecule release may be preferable over CW laser-induced drug release for certain applications, for instance, those performed in situ.

After employing either CW or pulsed laser irradiation to trigger guest molecule release, allowing a treatment period to pass may give the guest molecules time to produce a desired effect. For example, in some embodiments, when the guest molecule is a drug, allowing a treatment period may give the drug time to interact with its intended target. This treatment period may provide the time necessary for a chemotherapy drug to induce cell death. In some embodiments, this treatment period may vary depending on the guest molecule and the intended effect. In some embodiments, the treatment period may range from minutes to weeks or longer (e.g., at least 1 min; at least 3 min; at least 5 min; at least 10 min; at least 15 min; at least 30 min; at least 1 hr.; at least 2 hr.; at least 5 hr.; at least 12 hr.; at least 18 hr.; at least 1 day; at least 2 days; at least 3 days; at least 5 days; at least 1 week; at least 2 weeks; at least 1 month; etc.).

In view of the above, one having skill in the art will readily appreciate the innumerable combinations of nanoshells, host molecules, and guest molecules that may be used to create a nanocomplex according to one or more embodiments of this disclosure. Similarly, a person of skill in the art will also recognize the applicability of the method for creating the large variety of compositions for said nanocomplex.

Furthermore, a person of ordinary skill will appreciate that the combination of host molecules and guest molecules may be chosen together in order to interact synergistically. For example, as discussed above, a hydrophobic guest molecule may benefit from a host molecule having an outwardly hydrophobic character. Furthermore, the host molecule may aid in preventing the guest molecule from interacting with the body prior to being intentionally released. Once released, the host molecule may not impact the activity of the guest molecule, but prior to release the host molecule may inhibit the activity of the guest molecule. Additional features beyond the hydrophobicity and/or activity of the host/guest pair may also be important.

To further elucidate one or more embodiments and to guide a skilled practitioner in applying the embodiments described herein, following are a few illustrative examples of both nanosamples and the methods for producing said nanosamples, according to one or more embodiments.

### Example 1: NS @dsDNA@DTX Nanosample

In one or more embodiments of the invention, a nanosample that includes the chemotherapy agent DTX is produced using a four-step process including: synthesizing a gold nanoshell; functionalization of a surface of the nanoshell with dsDNA to form a nanohost; incubating the NS @dsDNA nanohost in a solution containing DTX to form a nanocomplex; and sterilizing and concentrating the NS @dsDNA@DTX nanocomplex solution to form a nanosample. An additional step may be performed, in some embodiments, to purify and hybridize the single strand DNA (ssDNA) to prepare and form the dsDNA used to functionalize the NS. Additional details about the four-step process are included below.

Synthesis of a Nanoshell

In one or more embodiments, nanoshells may have a silica core and a gold shell with core-shell dimensions of [r1, r2]=[60,86] nm. Said nanoshells may have a nanoshell plasmon resonance at ˜770 nm in aqueous solution. The nanoshells may be synthesized according to a previously published procedure.

Purification and Hybridization of ssDNA

In some embodiments, a thiolated ssDNA and an un-thiolated ssDNA strand may be hybridized to form a dsDNA that may be linked to the NS. In one or more embodiments, a DNA sequence of the thiolated ssDNA and the un-thiolated ssDNA may be complementary, and thus form the dsDNA used below. In some embodiments, a thiolated ssDNA may be purified prior to combination with an un-thiolated ssDNA to create the dsDNA that is ultimately linked to the NS. Alternatively, some embodiments of this disclosure may not require said purification and/or hybridization to prepare the dsDNA feed stock.

In one or more embodiments, the two oligonucleotide sequences that make up the dsDNA may be 22-bp ssDNA 5′-HS-C6H12-GGA ATA CAC GCG CGA AAT CAC G-3′ (SEQ ID NO: 1) (the thiolated ssDNA) and the complementary sequence 5′-CGT GAA TTC GCG CGT GTA TTC C-3′ (SEQ ID NO: 2) (the un-thiolated ssDNA). This dsDNA sequence is referred to as DNA1 in the below discussion and in the figures.

In some embodiments, the thiolated ssDNA may be incubated with 10 mM dithiothreitol (DTT) reducing agent in a 10 mM TE buffer. TE buffer is a mixture of tris(hydroxymethyl)aminomethane (Tris) and ethylenediaminetetraacetic acid (EDTA). The thiolated ssDNA may be allowed to react with DTT for 1 h at room temperature, to allow the reduction of the disulfide bonds. This reaction may allow the reduction of disulfide bonds in preparation for attachment of the dsDNA to the NS.

In some embodiments, a desalting column may be eluted with TE buffer and/or water before use. In some embodiments, any resulting salts and other impurifies may be removed from the thiolated ssDNA using a number of well-known purification techniques, including use of a filtration gel (e.g., SEPHADEX G-25) or commercial gel filtration column (e.g., GE HEALTHCARE ILLUSTRA NAP-25) or other methods.

In some embodiments, the un-thiolated ssDNA strands may be dispersed in 600 μL TE buffer without further purification.

To form the dsDNA, according to one of more embodiments, the two complementary ssDNA samples may be mixed in a 1:1 molar ratio in a TE buffer solution with 33 mM sodium chloride (NaCl), heated at 100° C. in a large water bath for 4 min, and then cooled slowly overnight to room temperature. The oligonucleotide concentrations of both the thiolated and un-thiolated ssDNA may be determined using a ultraviolet-visible spectroscopy (UV-vis) spectrophotometer or by some other method known in the art.

Functionalization of a Nanoshell with dsDNA

In some embodiments, oligonucleotide sequences of the dsDNA may be 22-base pair (bp) ssDNA 5′-HS-C6H12-GGA ATA CAC GCG CGA AAT CAC G-3′ (SEQ ID NO: 1) and the complementary sequence 5′-CGT GAA TTC GCG CGT GTA TTC C-3′ (SEQ ID NO: 2). This sequence is a variation of Dickerson's dodecamer, d(CGCGAATTCGCG)2 (SEQ ID NO: 6), which complexes taxol and other analogs. The base substitutions were made to result in the dsDNA structure having a higher melting point than any hairpin structures, which allows for preferential formation of the DNA duplex upon DNA cooling in the hybridization process. This dsDNA sequence is referred to as DNA1 in the below discussion and in the figures.

In one or more embodiments, dsDNA may be attached to a surface of the gold nanoshell via thiol-gold interaction by overnight functionalization of aqueous NS with dsDNA in an excess of 20,000 dsDNA strands/NS, forming a nanohost. In some embodiments, the aqueous NS and dsDNA solution may be stirred during functionalization.

After functionalization, the nanohost in solution may be centrifuged twice at 300 g for 20 min to remove any unbound dsDNA strands and may be redispersed in TE buffer at pH 7.5.

Incubation of NS @DNA1 with DTX

In some embodiments, the NS @DNA1 host molecule may be incubated with DTX at a ratio of 3 drug molecules per dsDNA strand, assuming 20,000 dsDNA strands/NS (˜20 nM DTX), on an orbital shaker at 175 rpm overnight to form a nanocomplex.

In one or more embodiments, after incubation, the NS @DNA@DTX may be centrifuged three times at 300 g for 20 min and resuspended in a minimal volume of TE buffer, pH 7.5.

In some embodiments, the NS @DNA1 @DTX nanocomplexes may be diluted to a final concentration of 2×10⁸ NS/mL with DMEM media. DMEM (Dulbecco's Modified Eagle Medium) is a broadly suitable medium for many adherent cell phenotypes.

Sterilizing and Concentrating the Nanocomplex Solution

In some embodiments, the nanocomplex solution may be sterilized by being passed through a biological cellular filter. A 0.8/0.2 μm pore size syringe filter may be used, in one or more embodiments.

In some embodiments, the nanocomplex solution may be centrifuged at 280 rcf for 30 min. In some embodiments, the final solvent of the nanosample may be sterile 1 mM phosphate buffer, pH 7.3. In some embodiments, the final concentration of the nanosample may be 1.5 mg of Au/mL.

### Example 2: NS @HSA @LAP Nanosample

In one or more embodiments of the invention, a nanosample that includes the chemotherapy agent LAP is produced using a four-step process including: synthesizing a gold nanoshell; functionalization of a surface of the nanoshell with HSA to form a nanohost; incubating the NS @HSA nanohost in a solution containing LAP to form a nanocomplex; and sterilizing and concentrating the NS @HSA@LAP nanocomplex solution to form a nanosample. Further, in some embodiments, the HAS solution may require additional preparation and/or filtration steps before attachment to the NS as described below. Additional details about the four-step process is included below.

Synthesis of a Nanoshell

In one or more embodiments, nanoshells may have a silica core and a gold shell with core-shell dimensions of [r1, r2]=[60, 86] nm. Said nanoshells may have a nanoshell plasmon resonance at ˜770 nm in aqueous solution. The nanoshells may be synthesized according to a previously published procedure or may be purchased. In some embodiments, the nanoshells may be in a solution having a concentration of 10¹⁰ NS/mL.

HSA Solution Preparation

In one or more embodiments, a 200 μg/mL solution of HSA in 10 mM phosphate-buffered saline (PBS) at a pH 7 may be rocked for 30 min to dissolve HSA. Additionally, in some embodiments, the HSA solution may then be filtered, for example, using a 0.8/0.2 μm PES (hydrophilic polyethersulfone) filter.

Functionalization of a Nanoshell with HSA

In some embodiments, a solution of 800 μL filtered HSA and 800 μL PBS may be pre-heated to 37° C. In one or more embodiments, 500 μL of the NS were added to the solution and stirred for 1 h at 37° C. to form a nanohost via functionalization. In some embodiments, the nanohost may be centrifuged twice at 200 g for 15 min, resuspended in PBS, and/or filtered through a 0.8/0.2 μm PES filter.

Incubation of NS @HSA with LAP

In some embodiments, the NS @HSA nanohost may be incubated with 20,000 LAP/NS (˜6.6 nM) and rocked overnight. The resulting NS @HSA@LAP nanocomplex solution may be centrifuged three times at 300 g for 10 min, resuspended in PBS, and filtered.

In some embodiments, the NS @HSA@LAP nanocomplex may be diluted to a final concentration of 2×10⁸ NS/mL with DMEM media to form the nanosample.

Sterilizing and Concentrating the Nanocomplex Solution

In some embodiments, the nanocomplex solution may be sterilized by being passed through a biological cellular filter. A 0.8/0.2 μm pore size syringe filter may be used, in one or more embodiments.

In some embodiments, the nanocomplex solution may be centrifuged at 280 rcf for 30 min. In some embodiments, the final solvent of the nanosample may be sterile 1 mM phosphate buffer, pH 7.3. In some embodiments, the final concentration of the nanosample may be 1.5 mg of Au/mL.

### Example 3: NS @DNA2 @LAP Nanosample

A nanocomplex with an NS @DNA2@LAP structure, according to one or more embodiments, may be prepared very similarly to Example 1 above.

In some embodiments, dsDNA is first attached to NS through Au-thiol bonds.

One difference is that the two ssDNA oligonucleotide sequences used for the LAP-containing nanocomplex may be 27-bp ssDNA 5′-HS-C6H12-AAA AAA ATA TAT AAT TAA AAG TTG AAA-3′ (SEQ ID NO: 3) (the thiolated ssDNA) and the complementary sequence 5′-TTT TTT TAT ATA TTA ATT TTC AAC TTT-3′ (SEQ ID NO: 4) (the un-thiolated ssDNA). An adenine-thymine-rich sequence was chosen for LAP. LAP's structure contains a pyrimidinamine, analogous to adenine, and thus binds to thymine in a similar manner. This dsDNA sequence is referred to as DNA2 in the below discussion and in the figures.

### Example 4: NS @ DNA3 @ TNF-α

In one or more embodiments of the invention, a nanosample that includes the protein Tumor Necrosis Factor alpha (TNF-α) may be produced using a four-step process including: synthesizing a gold nanoshell; functionalization of a surface of the nanoshell with the TNF-α aptamer (VR11) to form a nanohost; incubating the NS @DNA nanohost in a solution containing TNF-α to form a nanocomplex; and sterilizing and concentrating the NS @DNA@TNF-α nanocomplex solution to form a nanosample. Further, in some embodiments, the TNF-α solution may require additional preparation and/or filtration steps before attachment to the NS as described below. Additional details about the four-step process is included below.

Synthesis of a Nanoshell

In one or more embodiments, nanoshells may have a silica core and a gold shell with core-shell dimensions of [r1, r2]=[60, 75] nm. The nanoshells may be synthesized according to a previously published procedure or may be purchased.

Purification and Hybridization of ssDNA

Prior to attachment to the nanoshell surface, thiolated ssDNA [HS-C6H12-TGG TGG ATG GCG CAG TCG GCG ACA A] (SEQ ID NO: 5) may be incubated for 1 h at room temperature with 10 mM threo-1,4-dimercapto-2,3-butanediol (a DTT reducing agent) prepared in 10 mM Tris, pH 7.5, 0.1 mM EDTA buffer (1×TE Buffer) in order to remove the protecting group from the oligomers. Excess and oxidized DTT may be removed by passing the solution through a NAP 25 gel filtration column. Elution of the column with nuclease-free water may be performed to collect the purified ssDNA in water.

Functionalization of a Nanoshell with ssDNA

In some embodiments, oligonucleotide sequences of the ssDNA may be 25-bp ssDNA

HS-C₆H₁₂-TGG TGG ATG GCG CAG TCG GCG ACA A (SEQ ID NO: 5). This ssDNA sequence is referred to as DNA3 in the below discussion and in the figures.

In some embodiments, thiolated ssDNA may be attached to the NS surface by mixing 300 μL of 4 μM ssDNA with an aqueous suspension of NS (7 mL; 1×10¹⁰ particles/mL). To ensure maximum NS surface coverage, a small volume of 100 mM pH 3 formate buffer may be rapidly added to the NS @DNA3 nanohost followed by gentle vortexing, yielding a final formate buffer concentration of ˜25 mM. The NS @DNA3 nanohost may be allowed to react overnight with continuous shaking. The solution may be washed three times by centrifugation and re-suspended in ultrapure water to remove excess ssDNA and ultimately may be resuspended in 1 mM phosphate buffer at pH 7.4.

Incubation of NS @DNA3 with TNF-α

10 mL of 1×10¹⁰ particles/mL NS @DNA may be incubated with 164 μL, 5.7 μM TNF-α for 3 hours on ice using an orbital shaker. Excess protein may be removed by three rounds of centrifugation and rinsing with ultrapure water.

Sterilizing and Concentrating the Nanocomplex Solution

In some embodiments, the nanocomplex solution may be sterilized by being passed through a biological cellular filter. A 0.8/0.2 μm pore size syringe filter may be used, in one or more embodiments.

In some embodiments, the nanocomplex solution may be centrifuged at 280 rcf for 30 min. In some embodiments, the final solvent of the nanosample may be sterile 1 mM phosphate buffer, pH 7.4. In some embodiments, the final concentration of the nanosample may be 1×109 particles/mL.

Nanocomplex Characterization

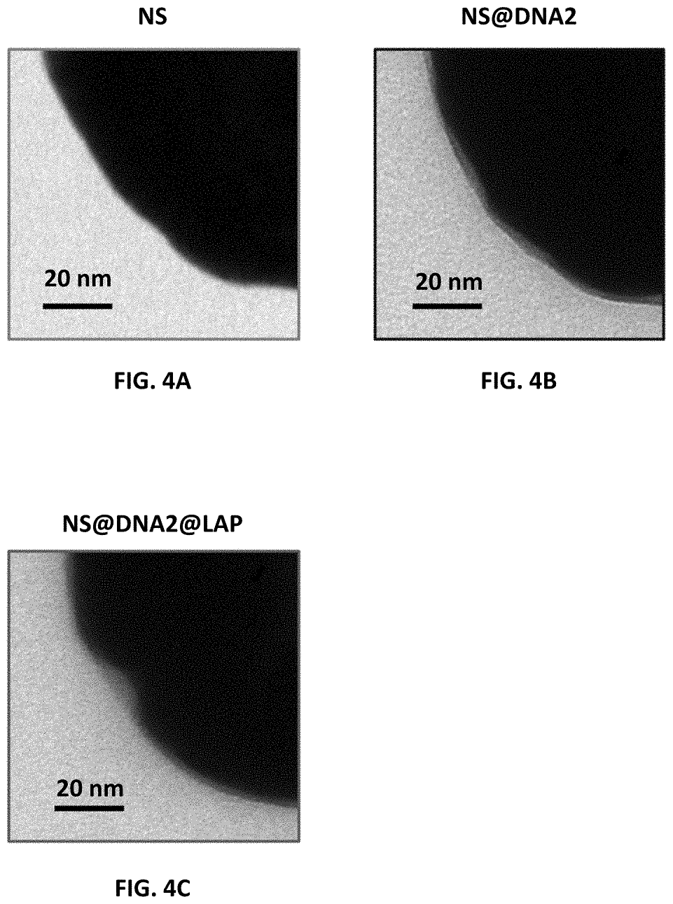

FIGS. 4A-4C depict transmission electron microscopy (TEM) images of an NS @DNA2@LAP nanocomplex at various steps during formation, according to one or more embodiments. The scale bar in each image indicates 20 nm. According to one or more embodiments, FIG. 4A depicts bare, spherical NS particles with smooth regular surfaces;

FIG. 4B depicts an NS @DNA2 nanohost including the formation of a thin dsDNA layer around the NS; and FIG. 4C depicts an NS @DNA2@LAP nanocomplex showing no morphological changes compared with the NS @DNA2 nanohost. Therefore, comparing FIG. 4A with FIGS. 4B and 4C, the formation of a thin DNA layer around the NS may be seen.

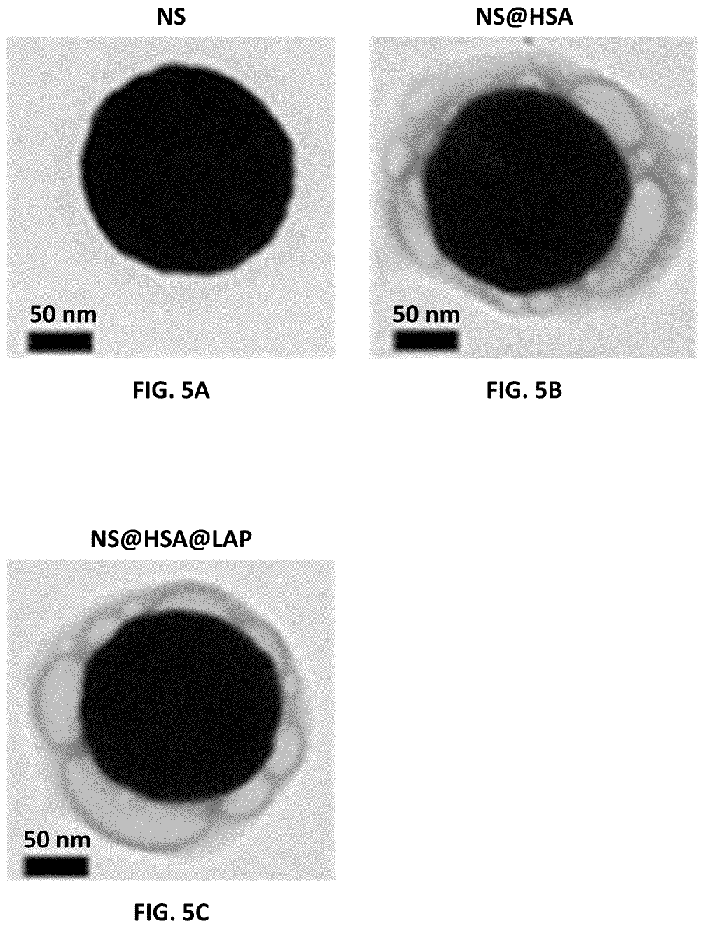

FIGS. 5A-5C depict TEM images of an NS @HSA@LAP nanocomplex at various steps during formation, according to one or more embodiments. The scale bar in each image indicates 50 nm. FIG. 5A depicts bare NS; FIG. 5B depicts an NS @HSA nanohost; and FIG. 5C depicts an NS @HSA@LAP nanocomplex, according to one or more embodiments. Comparing FIG. 5A with FIGS. 5B and 5C, a hard and a soft corona around the NS may be seen.

Table 1 indicates the extinction maxima, the zeta-potential, and the hydrodynamic diameter of a number of nanocomplexes and their intermediate constituents, according to one or more embodiments. Extinction maxima were collected with a UV/Vis/NIR spectrophotometer; zeta-potential measurements were obtained using laser doppler micro-electrophoresis; and the hydrodynamic diameters were measured via dynamic light scattering. Changes in each of these metrics during formation of the nanocomplexes indicate that the guest molecule were incorporated into each of these nanocomplexes.

TABLE 1

| | Extinction | Zeta Potential | Hydrodynamic |
|---|---|---|---|
| Sample | Maximum (nm) | (mV) | Diameter (nm) |
| NS | 766 | −35.6 ± 0.3 | 178.7 ± 0.6 |
| NS@DNA1 | 770 | −46.2 ± 1.2 | 165.9 ± 1.2 |
| NS@DNA1@DTX | 774 | −62.5 ± 0.8 | 155.4 ± 2.2 |
| NS | 771 | −36.4 ± 0.9 | 176.1 ± 1.8 |
| NS@DNA2 | 774 | −42.2 ± 1.2 | 168.7 ± 1.4 |
| NS@DNA2@LAP | 779 | −55.0 ± 1.1 | 163.7 ± 1.1 |
| NS | 771 | −35.3 ± 0.5 | 177.5 ± 2.0 |
| NS@HSA | 788 | −35.5 ± 0.9 | 180.8 ± 2.2 |
| NS@HSA@LAP | 786 | −31.4 ± 0.6 | 178.9 ± 4.0 |

First, the extinction maxima of the NS red-shift when linked with dsDNA and further red-shift when the guest molecules are linked with the dsDNA. This red-shift may be due to the changes in the dielectric environment around the NS. HSA coating induces a larger red-shift relative to dsDNA which may result from the larger size of the HSA layer. No changes in the peak shape or width of the extinction spectrum is observed, which may indicate that the nanocomplexes do not aggregate during the functionalization process.

Further, zeta-potential measurements further confirm modification of the NS surface. For example, the zeta potential became more negative after dsDNA attachment and even more negative with guest molecule loading.

Finally, dynamic light scattering (DLS) measurements show the hydrodynamic diameter of bare NS decreases after DNA functionalization, possibly due to a change in the solvent from water to TE buffer. The hydrodynamic diameter further decreases after guest molecule loading, likely as a result of compaction of the DNA. In the case of protein functionalization, the hydrodynamic diameter increases slightly after protein coating and shows minimal change upon drug loading.

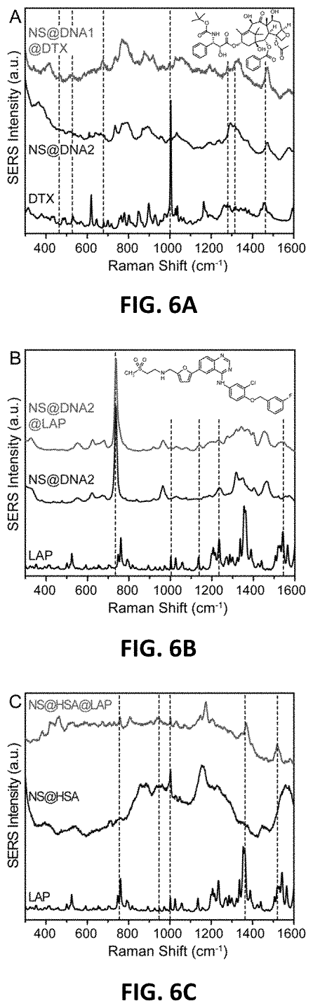

FIGS. 6A-6C show surface-enhanced Raman spectroscopy (SERS) spectra for Examples 1-3, which was used to confirm drug loading of the NS @HostMolecule complexes, according to one or more embodiments.

FIG. 6A compares the intermediate and final components of the NS @DNA1 @DTX nanocomplex (Example 1), according to one or more embodiments. The SERS spectrum of the NS @DNA1 nanohost shows several characteristic SERS bands at 1,346 and 1,480 cm⁻¹. Several strong SERS peaks at 1,003, 1,271, 1,520, and 1,543 cm⁻¹ assigned to the benzene ring modes (vC-C, vC-O, vC=C, and ring stretching) are seen in the NS @DNA1@DTX SERS spectrum. This indicates DNA1-DTX link formation. Additionally, the bands at 1,340 and 1,475 cm⁻¹ are enhanced in the NS @DNA1 @DTX nanocomplex spectrum in comparison with the NS @DNA1 nanohost. The SERS mode at 737 cm⁻¹ is weak in the NS @DNA1 due to the low number of adenine bases in the DNA sequence.

FIG. 6B compares the intermediate and final components of the NS @DNA2@LAP nanocomplex (Example 2), according to one or more embodiments. In the case of the NS @DNA2 monolayer on the NS surface, the SERS spectrum is dominated by the adenine ring-breathing mode at 737 cm⁻¹.

FIG. 6C compares the intermediate and final components of the NS @HSA@LAP nanocomplex (Example 3), according to one or more embodiments. LAP Raman peaks including C—O—C mode of a substituted furan at 1,136 cm⁻¹ are seen in the NS @DNA2@LAP SERS spectra. The protein-drug nanocomplex SERS data, including a quinazoline ring stretch at 1,369 cm⁻¹, confirm that LAP has linked to the NS @HSA nanohost.

Guest Molecule Release

According to one or more embodiments, remotely triggering guest molecule release using a CW laser irradiation may involve irradiating a nanosample with an 808 nm continuous wave (CW) laser at 1.5 W or at 2.0 W for 2 min.

In some embodiments, remotely triggering guest molecule release using a CW laser irradiation may involve irradiating a nanosample with an 808 nm CW laser having a CW laser power and a CW laser duration optimized such that a final solution temperature may be greater than a threshold temperature. In some embodiments, the threshold temperature of a nanosample may be determined experimentally. In some embodiments, the threshold temperature may be 55° C.

Furthermore, according to one or more embodiments, remotely triggering guest molecule release using a pulsed laser may involve irradiating a nanosample with an 800 nm, 160 fs pulsed laser at 25 mW for 2 min.

Treatment of Breast Cancer Cells with Various Nanocomplexes

Cellular uptake of the NS @HostMolecule@GuestMolecule complexes within the cell boundary around but not inside the nucleus was confirmed in RAW 264.7 macrophage cells (healthy cells used as a control), and in MDA-MB-231-eGFP and SKBR3 breast cancer cells via multiple light microscopy methods and TEM.

Cellular cytotoxicity resulting from light-triggered release of DTX and LAP from both dsDNA and HSA nanohost molecules was evaluated. Cells were irradiated, according to one or more embodiments, with either a CW laser imparting 1.5 W of power or with a pulsed-laser using a Ti:Sapphire tunable laser operating at 250 kHz with an average power of 25 mW and 160-fs pulsed-laser power for 2 min.

After the guest molecules were released, each sample was treated at 37° C., 5% CO₂ for a treatment period of 24 h to allow time for the released drug guest molecules to interact with the cells. Cell death was evaluated with a lactate dehydrogenase (LDH) cytotoxicity assay. Percent cytotoxicity values were calculated considering 10% cell death in the control experiment, NS @host with no laser treatment.

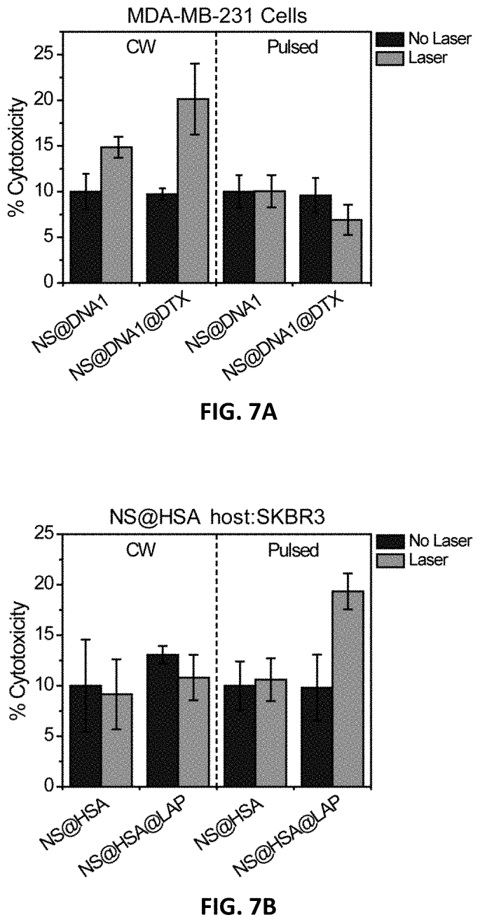

FIG. 7A depicts results for an NS @DNA1 @DTX nanocomplex and an NS @DNA1 nanohost before and after irradiation with either NIR CW or pulsed laser irradiation.

The NS @DNA1 @DTX nanocomplex is shown to be nontoxic, since statistically no increase in cytotoxicity was observed compared with the NS @DNA1 nanohost. This indicates that DTX stays confined within the NS @DNA1 nanohost and that no leaching occurs. A very statistically significant (P<0.01) increase in cytotoxicity for the cells with NS @DNA1 after CW is likely due to photothermal ablation.

FIG. 7A shows a statistically significant (P<0.05) increase in breast cancer cell death under CW laser irradiation between the NS @DNA1 and the NS @DNA1 @DTX nanocomplex, confirming there is a positive effect of DTX release beyond any photothermal heating caused by the laser. Additionally, a very statistically significant (P<0.01) increase in cell death is also seen in FIG. 7A, illustrating that when DTX is released from the NS @DNA1 @DTX nanocomplex via the CW laser the DTX causes additional cell death compared with the NS @DNA1 @DTX without laser irradiation.

No increased cell death is seen for pulsed laser-induced release of DTX in FIG. 7A. This is most likely due to the different DNA release mechanisms resulting from CW vs. pulsed lasers. In the case of a CW laser, the nanocomplex and its surrounding local environment undergo a photothermal temperature increase, causing the dsDNA to de-hybridize, releasing the drug and ssDNA. In contrast, irradiation with a pulsed laser induces breakage of the Au—S anchoring bond of the dsDNA. As a result, the entire dsDNA-drug complex is released, with the drug likely remaining sequestered within the DNA. This structure may restrict DTX from inducing cell death upon irradiation.

Similar cytotoxicity results are seen for both the MDA-MB-231 breast cancer and the noncancerous macrophage cells, which are to be expected, because DTX is not a targeted cancer drug that does not discriminate between cancerous and healthy cells.

LAP was chosen to see if cell death could be preferentially induced in cancer cells over healthy cells, since LAP specifically targets the HER2 receptor that is overexpressed on SKBR3 cells. LAP was released from both an NS @DNA nanohost control and an NS @HSA nanohost in both SKBR3 breast cancer cells and RAW 264.7 macrophage cells.

For the NS @DNA nanohost, no statistically significant increase in cytotoxicity is observed for either CW or pulsed-laser-induced release for SKBR3 cells.

FIG. 7B shows there is also no increase in cytotoxicity for CW laser-induced release of LAP from the NS @HSA@LAP nanocomplex; however, there is a very statistically significant (P<0.01) increase in cytotoxicity for pulsed-laser induced LAP release from the NS @HSA@LAP nanocomplex in SKBR3 cells.

The low average power (25 mW) of the pulsed-laser does not increase the local temperature and, therefore, cells with the NS @HSA nanohost do not show increased death after pulsed-laser treatment. However, pulsed-laser irradiation is clearly sufficient to alter the HSA, releasing LAP from the NS @HSA@LAP nanocomplex, shown by the extremely statistically significant (P<0.001) increase in cytotoxicity vs. pulsed irradiation of NS @HSA without LAP.

LAP release was performed in the noncancerous macrophage cells as a control. Because they lack the HER2 receptor, LAP does not cause cytotoxicity in macrophages. No increase in cytotoxicity due to LAP release from the NS @HSA@LAP nanocomplex by either CW or pulsed-laser was observed, although the CW caused nonspecific cell death. The lack of increased cytotoxicity after LAP release from the NS @HSA@LAP nanocomplex via pulsed laser irradiation confirms that the laser treatment did not directly induce the cell death caused by LAP release from the NS @HSA nanohost within SKBR3 cells. Thus, the released LAP selectively induces cell death in HER2 expressing breast cancer cells, without affecting noncancerous cells.

What is claimed is:

1. A nanosample comprising:
a plurality of nanocomplexes, each nanocomplex comprising:
a nanoshell, comprising:
a shell;
a host molecule linked to the nanoshell; and
a guest molecule linked to the host molecule,
wherein the nanocomplex has a plasmon resonance wavelength.

2. A nanosample capable of near-infrared light-triggered release of therapeutic molecules, the nanosample comprising:
a plurality of nanocomplexes, each nanocomplex comprising:
a nanoshell, comprising:
a shell;
a host molecule linked to the nanoshell; and
a guest molecule linked to the host molecule,
wherein the nanocomplex has a plasmon resonance wavelength, and
wherein, when irradiated with electromagnetic radiation of the plasmon resonance wavelength, plasmon resonance of the nanocomplex releases the guest molecule.

3. The nanosample of claim 2,
wherein the nanoshell further comprises a core, and
wherein the shell surrounds the core.

4. The nanosample of claim 2,
wherein the nanoshell is a nanomatryoshka.

5. The nanosample of claim 2,
wherein a link between the nanoshell and the host molecule is a gold-thiol interaction.

6. The nanosample of claim 2,
wherein the shell comprises at least one metal.

7. The nanosample of claim 6, wherein:
the core comprises at least one of a liposome or silica; and
the shell comprises at least one of gold or silver.

8. The nanosample of claim 2,
wherein the host molecule comprises at least one of synthetic polymers, biopolymers, polynucleotides, nucleic acids, polypeptides, polysaccharides, polyterpenes, lipids, aptamers, proteins, or combinations thereof.

9. The nanosample of claim 2,
wherein the guest molecule comprises at least one of pharmaceutical molecules, biopharmaceutical molecules, oligonucleotides, nucleic acids, dye molecules, imaging contrast agents, or combinations thereof.

10. The nanosample of claim 8,
wherein the host molecule is selected from tumor necrosis factor alpha (TNF-α) aptamer, single-stranded DNA (ssDNA), double-stranded DNA (dsDNA), or human serum albumin (HAS).

11. The nanosample of claim 9,
wherein the guest molecule is selected from docetaxel (DTX), lapatinib (LAP), and TNF-α.

12. The nanosample of claim 2,
wherein the host molecule is dsDNA and the guest molecule is DTX.

13. The nanosample of claim 2,
wherein the host molecule is HAS and the guest molecule is LAP.

14. The nanosample of claim 2,
wherein the host molecule is TNF-α aptamer and the guest molecule is TNF-α.

15. A method for fabricating a nanosample capable of near-infrared light-triggered release of therapeutic molecules, the method comprising:
synthesizing a nanoshell;
functionalizing a surface of the nanoshell with a host molecule to form a nanohost;
incubating the nanohost with a guest molecule to form a nanocomplex; and
sterilizing the nanocomplex and forming the nanosample.

16. The method of claim 15,
wherein the host molecule comprises at least one of synthetic polymers, biopolymers, polynucleotides, nucleic acids, polypeptides, polysaccharides, polyterpenes, lipids, aptamers, proteins, or combinations thereof.

17. The method of claim 15,
wherein the guest molecule comprises at least one of pharmaceutical molecules, biopharmaceutical molecules, oligonucleotides, nucleic acids, dye molecules, imaging contrast agents, or combinations thereof.

18. The method of claim 16,
wherein the host molecule is selected from TNF-α aptamer, ssDNA, dsDNA, or HAS.

19. The method of claim 17,
wherein the guest molecule is selected from DXT, LAP, or TNF-α.

20. The method of claim 16,
wherein the host molecule is dsDNA and the guest molecule is DTX.

21. The method of claim 16,
wherein the host molecule is HAS and the guest molecule is LAP.

22. The method of claim 16,
wherein the host molecule is TNF-α aptamer and the guest molecule is TNF-α.

23. The method of claim 15, further comprising
preparing the host molecule for functionalization of the nanoshell by:
reducing the disulfide bonds of two ssDNA samples, and
mixing the two ssDNA samples to form dsDNA,
wherein the host molecule is the dsDNA, and
wherein the two ssDNA samples have complementary sequences.

24. A nanosample capable of near-infrared light-triggered release of therapeutic molecules for treating cancer, the nanosample comprising:
a plurality of nanocomplexes, each nanocomplex comprising:
a nanoshell, comprising:
a shell comprising a metal; and
a core comprising a non-metal;
a host molecule linked to the nanoshell; and
a guest molecule linked to the host molecule,
wherein the host molecule is selected from an aptamer, ssDNA, dsDNA, or HAS,
wherein the guest molecule is a pharmaceutical molecule or a biopharmaceutical molecule,
wherein the nanocomplex has a plasmon resonance wavelength in a near-infrared (NIR) water window, and
wherein the nanocomplex releases the guest molecule when irradiated with electromagnetic radiation of the plasmon resonance wavelength.
